(19) United States
(12) Patent Application Publication
(10) Pub. No.: US 2025/0011762 A1
(43) Pub. Date: Jan. 9, 2025

(54) LIBRARIES OF NUCLEIC ACIDS AND METHODS FOR OPTIMIZATION OF MRNA

(71) Applicant: QUERDENKER APS, Årslev (DK)

(72) Inventor: Thorleif MØLLER, Årslev (DK)

(73) Assignee: QUERDENKER APS, Årslev (DK)

(21) Appl. No.: 18/712,106

(22) PCT Filed: Nov. 24, 2022
(86) PCT No.: PCT/EP2022/083213

§ 371 Date: May 21, 2024

(30) Foreign Application Priority Data
Nov. 24, 2021 (DK) PA202101118

(51) Int. Cl. C12N 15/10; C40B 40/08

(52) U.S. Cl. CPC C12N 15/1093; C12N 15/1096; C40B 40/08

(57) ABSTRACT

The present invention provides libraries of nucleic acids, wherein each nucleic acid specie of the libraries comprises a unique sequence of triplet codons encoding the same polypeptide and methods of using such libraries for identification of mRNA encoding specific polypeptides with improved characteristics e.g., with regards to improved chemical stability and improved biostability, while still allowing for efficient translation in target cells.

### BACKGROUND OF THE INVENTION

There is great interest in the development of messenger RNA (mRNA) as therapeutics and vaccines. However, mRNAs suffer from various drawbacks such as poor stability in the human body and very limited shelf life. mRNA is also very poorly taken up by cells. Thus, there is a need for methods for improving mRNA.

### SUMMARY OF THE INVENTION

The present invention provides libraries of nucleic acids, wherein each nucleic acid specie of the libraries comprises a unique sequence of triplet codons encoding the same polypeptide and methods of using such libraries for identification of mRNAs encoding specific polypeptides with improved characteristics e.g., with regards to improved chemical stability, while still allowing for efficient translation in target cells.

### DISCLOSURE OF THE INVENTION

A “polypeptide” as used herein is a sequence of amino acids, i.e. a protein is herein also denoted a polypeptide. It is preferred that the polypeptide is of human origin. Also, polypeptides that are derived from bacteria and virus are preferred. Preferred polypeptides are those that may be used to elicit a therapeutic effect. Also, polypeptides that may be used to elicit an immune response are preferred, e.g. polypeptides originating from virus or bacteria or polypeptides that may be used to elicit an immune response against cancer cells. A particular preferred polypeptide is the spike protein of SARS-COV-2.

A “conservative amino acid substitution” as used herein is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative.

### FIRST ASPECT—LIBRARY OF NUCLEIC ACID SPECIES

In a first aspect, the present invention provides a library of nucleic acid species, wherein at least 0.1% of the nucleic acid species comprises a unique sequence of triplet codons encoding the same polypeptide or a conservatively substituted version of the polypeptide.

When referring to a nucleic acid specie, what is meant is a nucleic acid specie with a unique sequence in the library. A library of 10³ thus includes 10³ different nucleic acid sequences. As used herein, two nucleic acid sequences that differ in sequence by at least 1 nucleotide are herein referred to as two nucleic acid species.

In other embodiments of the library, at least 0.000001%, 0.00001%, 0.0001%, 0.001%, 0.01%, 0.5%, 1%, 5%, 10%, 20%, 30%, 40%, 50%, 60%, 70%, 80%, 90% or 95% of the nucleic acid species comprises a unique sequence of triplet codons encoding the same polypeptide or a conservatively substituted version of the polypeptide.

Even more preferably, 99% of the nucleic acid species encode the same polypeptide or a conservatively substituted version of the polypeptide, such as 99.9%, 99.99% or 100%.

In most embodiments, it is preferred that all nucleic acid species encode the same polypeptide or a conservatively substituted version of the polypeptide, although it is recognized that errors in library generation may lead to less than 100% of the polypeptide encoding the same polypeptide.

For all embodiments of the invention, it is preferred that the nucleic acid species encode the same polypeptide or a conservatively substituted version of the polypeptide and even more preferred is that the nucleic acid species encode the same polypeptide.

In preferred embodiments, the nucleic acid species are composed of either RNA such as mRNA or composed of DNA e.g., single stranded DNA or double stranded DNA.

Many different nucleic acid species can encode the same polypeptide because of the degenerate genetic code, wherein multiple synonymous triplet codons can encode the same amino acid. Thus, in a preferred embodiment, the nucleic acid species of the library differ in their use of (synonymous) codons for specific amino acids.

The term “encode” as used herein with reference to triplet codons, applies both for triplet codons in RNA and for triplet codons in DNA that may be converted to corresponding triplet codons in RNA using RNA transcription. Thus, triplet codons in DNA include T (thymidine) instead of U (uridine).

Synonymous codons for various amino acids and start and stop codons are listed here:

| | |
|---|---|
| Ala/A: | GCU, GCC, GCA, GCG |
| Arg/R: | CGU, CGC, CGA, CGG, AGA, AGG |
| Asn/N: | AAU, AAC |
| Asp/D: | GAU, GAC |
| Cys/C: | UGU, UGC |
| Gln/Q: | CAA, CAG |
| Glu/E: | GAA, GAG |
| Gly/G: | GGU, GGC, GGA, GGG |
| His/H: | CAU, CAC |
| Ile/I: | AUU, AUC, AUA |
| Leu/L: | UUA, UUG, CUU, CUC, CUA, CUG |
| Lys/K: | AAA, AAG |
| Met/M: | AUG |
| Phe/F: | UUU, UUC |
| Pro/P: | CCU, CCC, CCA, CCG |
| Ser/S: | UCU, UCC, UCA, UCG, AGU, AGC |
| Thr/T: | ACU, ACC, ACA, ACG |
| Trp/W: | UGG |
| Tyr/Y: | UAU, UAC |
| Val/V: | GUU, GUC, GUA, GUG |
| START: | AUG |
| STOP: | UAG, UGA, UAA |

Preferred synonymous codons are those known to give robust translation of mRNA in target cells or those with high levels of tRNAs in target cells. In one embodiment, poorly translated codons are not used.

Using synonymous codons in various positions in a nucleic acid such as an mRNA (or a portion of a mRNA), can create nucleic acid libraries of immense size. This is similar to RNA or DNA libraries created for SELEX or libraries for mRNA display or phage display, where experience shows that large libraries allow identification of new molecular function and activity. However, in these cases, the library members do not comprise a unique sequence of triplet codons encoding the same polypeptide. In the cases where such libraries (mRNA display and phage display libraries) comprise an open reading frame in the form of a sequence of triplet codons, the goal is most often not to conserve the amino acid (aa) sequence of the encoded polypeptide. On the contrary, the goal is to vary the sequence of triplet codons so that the aa sequence of the encoded polypeptide also varies. This is necessary to be able to identify new polypeptide sequences with desired characteristics, e.g., improved binding to a receptor or heat stability.

In the present invention, large libraries are used to search the vast structural space of nucleic acid species with sequences of triplet codons encoding the same polypeptide or a conservatively substituted version of the polypeptide. Given the sizes of libraries than can be generated as outlined in this specification, and that fragile regions in the mRNA reading frame are affected by the primary (sequence), secondary and tertiary structure, large libraries of mRNAs with triplet codons encoding the same polypeptide (or a conservatively substituted version of the polypeptide) will allow identification of mRNAs with improved characteristics, such as chemical stability thus improving shelf life of mRNA.

### Library Size and Copy Number

In one embodiment, the library includes at least 10³ different nucleic acid species, such as at least 10⁴, 10⁵, 10⁷, 10⁸, 10⁹, 10¹⁰, 10¹¹, 10¹², 10¹³, 10¹⁴ nucleic acid species.

In another preferred embodiment, the library includes at least 10³ different nucleic acid species, such as at least 101, 102, 10³, 10⁴, 10⁵, 10⁷, 10⁸, 10⁹, 10¹⁰, 10¹¹, 10¹², 10¹³ or 10¹⁴ nucleic species that all differ in their open reading frame, i.e. in their use of codons. And preferably, all encode the same polypeptide.

In yet another preferred embodiment, the library has a number of nucleic acid species (each) comprising a (unique) sequence of triplet codons selected from the group consisting of: at least 5, at least 10, at least 50, at least 102, at least 10³, at least 10⁴, least 10⁵, at least 106, at least 10⁷, at least 10⁸, at least 10⁹, at least 10¹⁰, least 10¹¹, at least 10¹², at least 10¹³, at least 10¹⁴, between 10 and 10¹⁴, between 102 and 10¹⁴, between 10³ and 10¹³, between 10⁴ and 10¹¹, between 10⁵ and 10¹⁰, between 106 and 10⁹, between 10³ and 10¹⁴, between 10⁴ and 10¹⁴, between 10⁵ and 10¹⁴, between 106 and 10¹⁴, between 10⁷ and 10¹⁴, between 10³ and 10¹², between 10⁴ and 10¹², between 10⁵ and 10¹², between 106 and 10¹², and between 10⁷ and 10¹².

The copy number of individual nucleic acid species may be adjusted as needed. In a preferred embodiment, the copy number of individual nucleic acid species in the library is between 1 and 10⁹, such as between 10 and 10⁸, between 102 and 10⁷, between 10³ and 106, and between 10⁴ and 10⁵. In another embodiment, the copy number of individual nucleic acid species in the library is at least 101, 102, 10³, 10⁴, 10⁵, 10⁷, 10⁸, 10⁹, 10¹⁰, 10¹¹, 10¹², 10¹³ or 10¹⁴.

In one embodiment, the copy number of each nucleic acid species of the library varies less than 10-fold or less than 9-fold, 8-fold, 7-fold, 6-fold, 5-fold, 4-fold, 3-fold or 2-fold. Favoured copy numbers depend on the library size, such that copy numbers for practical reasons can be bigger for smaller libraries than for large libraries.

### Length of Nucleic Acid Species

Preferably, the sequence of triplet codons has a length selected from the group consisting of: at least 3 nucleotides, at least 6 nucleotides, at least 9 nucleotides, at least 12 nucleotides, at least 15 nucleotides, at least 18 nucleotides, at least 21 nucleotides, 24 nucleotides, at least 27 nucleotides, at least 30 nucleotides, at least 33 nucleotides, at least 36 nucleotides, at least 39 nucleotides, at least 42 nucleotides, at least 45 nucleotides, at least 48 nucleotides, at least 51 nucleotides, 54 nucleotides, at least 57 nucleotides, at least 60 nucleotides, at least 75 nucleotides, at least 90 nucleotides, at least 105 nucleotides, at least 120 nucleotides, at least 150 nucleotides, at least 180 nucleotides, at least 210 nucleotides, at least 240 nucleotides, at least 270 nucleotides, at least 300 nucleotides, at least 600 nucleotides, at least 900 nucleotides, at least 1200 nucleotides, at least 1500 nucleotides, at least 1800 nucleotides, at least 2100 nucleotides, at least 2400 nucleotides, at least 2700 nucleotides, at least 3000 nucleotides, between 6 and 6000 nucleotides, between 60 and 6000 nucleotides, between 600 and 6000 nucleotides, between 600 and 6000 nucleotides, between 6 and 4500 nucleotides, between 60 and 4500 nucleotides, between 300 and 4500 nucleotides, and between 600 and 4500 nucleotides.

When the nucleic acid is RNA, it is preferred that the sequence of triplet codons has a length selected from the group consisting of: at least 30 nucleotides, at least 60 nucleotides, at least 90 nucleotides, at least 105 nucleotides, at least 120 nucleotides, at least 150 nucleotides, at least 180 nucleotides, at least 210 nucleotides, at least 240 nucleotides, at least 270 nucleotides, at least 300 nucleotides, at least 600 nucleotides, at least 900 nucleotides, at least 1200 nucleotides, at least 1500 nucleotides, at least 1800 nucleotides, at least 2100 nucleotides, at least 2400 nucleotides, at least 2700 nucleotides, at least 3000 nucleotides, from 60 to 6000 nucleotides, from 600 to 6000 nucleotides, from 6 to 6000 nucleotides, from 6 to 4500 nucleotides, from 60 to 4500 nucleotides, from 300 to 4500 nucleotides, and from 600 to 4500 nucleotides.

The length of polypeptides (encoded by the nucleic acid) may be from 10 amino acids (aa) to 10.000 aa. More preferably, the polypeptides have a length of at least 20 aa such as at least 30, 40, 50, 60, 70, 80, 90, 100, 200, 300, 400, 500, 600, 700, 800, 900, 1100, 1200, 1300, 1400 and 1500 aa.

Importantly, the nucleic acid species of the library does not necessarily encode a polypeptide that corresponds to a full-length naturally occurring protein. The nucleic acid species such as mRNA species may well encode only part of a naturally occurring protein, preferably a human, bacterial or viral protein. Moreover, the mRNA species of the library may not be well suited to be translated, because they lack certain elements that may be added later if or when translation is needed. The main thing is that the mRNA has a sequence of codons (open reading frame or ORF) corresponding to and encoding a polypeptide.

In a preferred embodiment, the sequence of triplet codons encoding the same polypeptide, encodes a naturally full length naturally occurring polypeptide.

### Fixed and Variable Regions

The sequence of triplet codons (and corresponding open reading frame) of nucleic acid species in the library may be divided into invariable regions and variable regions. And when reference is made to invariable and variable regions in relation to nucleic acid species in a nucleic acid library, invariable regions are those that are shared between all nucleic acid species of the library and variable regions are those that are not shared between all nucleic acid species of the library.

Invariable regions may also be said to be fixed regions or regions that are conserved between all nucleic acid species of the library. And likewise variable regions may also be said to be non-conserved regions that are not conserved between all nucleic acid species of the library.

In many cases, the sequence of triplet codons comprises both variable and invariable regions. In these cases, two different synonymous triplet codons are seen as a variable region of 3 nucleotides, even though only one nucleotide may differ between the two synonymous codons. That the sequence of triplet codons often comprises both variable and invariable regions is a consequence of the immense diversity possible by the use of synonymous codons. Even if using just the two best synonymous codons (e.g. corresponding to the most highly expressed tRNAs) for each amino acid, a sequence of 50 triplet codons will lead to a diversity of 2⁵⁰=1.13×10¹⁵. Thus, it is preferred that the total length of variable regions within the sequence of triplet codons is no longer than 150 nucleotides. It is even more preferred that the total length of variable regions within the sequence of triplet codons is no longer than 120 nucleotides, such as 105 nucleotides, 90 nucleotides, 75 nucleotides, 60 nucleotides, 45 nucleotides, 30 nucleotides, 15 nucleotides, 12 nucleotides, 9 nucleotides and 6 nucleotides.

The nucleotides outside of the sequence of triplet codons are most often not variable, although there are cases where it may be of interest to also create variable regions outside of the sequence of triplet codons, e.g. to stabilize the 5′UTR or 3′UTR of an mRNA or the combination of the 5′UTR or 3′UTR with the sequence of triplet codons.

Preferably, the nucleic acid species of the library share a first fixed sequence downstream of the sequence of triplet codons.

In one embodiment, the first fixed sequence comprises at least part of a sequence corresponding to the 3′UTR (3′untranslated region) of a naturally occurring mRNA. The first fixed sequence preferably has a length between 10 and 1000 nucleotides.

The term “corresponding to” as used herein means that the sequence is identical to, a DNA-version of an RNA-sequence or an RNA-version of a DNA-sequence.

In another embodiment, it is preferred that the nucleic acid species share a second fixed sequence upstream of the sequence of triplet codons.

The second fixed sequence may comprise at least part of a sequence corresponding to the 5′UTR (5′untranslated region) of a naturally occurring mRNA. And preferably, the length of the second fixed sequence is between 10 and 1000 nucleotides. In a preferred embodiment, the second fixed sequence comprises sequences corresponding to elements facilitating translation such as Kozak sequences, IRES elements (internal ribosome entry sites), 5′ CAP and a start codon.

In a preferred embodiment, the second fixed sequence comprises a promotor sequence for RNA transcription or a sequence corresponding to a promotor sequence. Preferred promotor sequences are those enable transcription with T7 RNA polymerase, SP6 RNA polymerase and Syn5 RNA polymerase.

In a preferred embodiment, the nucleic acid species of the library share both a first fixed sequence downstream of the sequence of triplet codons and a second fixed sequence upstream of the sequence of triplet codons.

When the library of the invention is composed of DNA, it is preferred that the second fixed sequence include a promotor enabling transcription of DNA sequences of triplet codons into corresponding RNA sequences of triplet codons.

When the library of the invention is composed of RNA, the second fixed sequence typically do not comprise a promotor enabling RNA transcription. However, a promotor sequence can be added by reverse transcription of the RNA into cDNA using a primer bound to the first fixed sequence and by second strand synthesis templated on the cDNA and primed with a primer containing a promotor sequence for RNA transcription in its 5′end and a sequence corresponding to the second fixed region in its 3′end end.

A Preferred Form of RNA is mRNA.

In such embodiment, the invention provides a library of mRNA species, wherein at least 50% of the mRNA species of the library encode the same polypeptide or a conservatively substituted version of the polypeptide. Even more preferably, at least 60%, 70%, 80%, 90% or 95% of the mRNA species encode the same polypeptide or a conservatively substituted version of the polypeptide. Even more preferably, 99% of the mRNA species encode the same polypeptide or a conservatively substituted version of the polypeptide, such as 99.9%, 99.99% or 100%.

In a more preferred embodiment, the present invention provides a library of mRNA species, wherein at least 50% of the mRNA species of library encode the same polypeptide. Even more preferably, at least 60%, 70%, 80%, 90% or 95% of the mRNA species encode the same polypeptide. Even more preferably, at least 99% of the mRNA species encode the same polypeptide, such as 99.9%, 99.99% or 100%.

mRNA species may comprise a 5′UTR and/or a 3′UTR. And the 5′UTR and 3′UTR regions may be conserved between mRNA species of the library. Alternatively, the 5′UTR and 3′UTR may differ between mRNA species of the library.

In some embodiments, the mRNAs species comprise elements necessary for facilitating translation such as kozak sequences, IRES elements, 5′ CAP and start codon, whereas in other embodiments such elements are not included.

In one embodiment, mRNA species are circularized before selecting for desired characteristics such as chemical stability.

Importantly, the ribonucleotides of the mRNA species of the library need not necessarily be limited to the four canonical ribonucleotides of RNA, adenine (A), guanine (G), cytosine (C), or uracil (U). In a preferred embodiment, pseudouridine or 1-methyl-3′-pseudouridylyl is included in the RNA or mRNA species and preferably instead of uridine. Also, other non-canonical ribonucleotides may be included such as e.g., 5-methylcytosine instead of cytosine. Phosphorothioate linkages may also be included.

### Libraries of Short Nucleic Acids

In a special embodiment, the nucleic acids species of the libraries of the invention are short and can be prepared with standard oligonucleotide synthesis. These libraries of short nucleic acids species can be used for assembling libraries of longer nucleic acids species with multiple variable regions in the sequence of triplet codons.

In this special embodiment, the first and second fixed sequence preferably each have a length between 10 and 50 nucleotides, more preferably between 12 and 40, 13 and 30 or 14 and 25 nucleotides. In this embodiment, it is also preferred that the nucleic acid species are single stranded DNA or double stranded DNA. Moreover, it is preferred that the sequence of triplet codons has a length selected from the group consisting of: between 3 and 300 nucleotides, between 3 and 150 nucleotides, between 3 and 120 nucleotides, between 3 and 90 nucleotides, between 3 and 60 nucleotides, between 3 and 45 nucleotides, between 3 and 30 nucleotides, between 3 and 15 nucleotides, between 6 and 300 nucleotides, between 6 and 150 nucleotides, between 6 and 120 nucleotides, between 6 and 90 nucleotides, between 6 and 60 nucleotides, between 6 and 45 nucleotides, between 6 and 30 nucleotides and between 6 and 15 nucleotides. And preferably, the number of nucleic acid species (each) comprising a (unique) sequence of triplet codon is selected from the group consisting of: at least 5, at least 10, at least 50, at least 100, at least 1000, between 3 and 1000, between 6 and 1000, between 6 and 500, between 6 and 300, between 6 and 100, between 6 and 50. Due to the relatively low number of nucleic acid species in this special embodiment, they may be synthesized individually and subsequently be mixed in equimolar amounts to create the library.

When libraries of short nucleic acids of the invention are to be used to assemble larger libraries of longer nucleic acids, a plurality of libraries of short nucleic acids are needed, wherein the nucleic acid species of each library of short nucleic acids has a sequence of triplet codons encoding the same polypeptide, but wherein the encoded polypeptide is not the same for different libraries of short nucleic acids. However, the polypeptides of the different libraries of short nucleic acids are preferably part of the same naturally occurring polypeptide.

### Creation of Libraries of the Invention

Libraries of nucleic acid species, either RNA or DNA, may be prepared using standard methods for RNA and DNA synthesis, e.g., based on phosphoroamidate building blocks. The nucleic acid species may be synthesised separately, and the species may subsequently be pooled. Chemical synthesis is particular relevant for libraries of nucleic acids species with a shorter length, such as less than 500 nucleotides, less than 400 nucleotides, less than 300 nucleotides, less than 200 nucleotides, less than 150 nucleotides or less than 100 nucleotides.

For mRNA libraries with longer mRNA species, enzymatic synthesis such as in vitro RNA transcription is a preferred method for creating the libraries. Thus, the mRNA species of the library of invention may be synthesized e.g., using SP6 RNA polymerase, T7 RNA polymerase or Syn5 RNA polymerase.

Obviously, RNA transcription requires a DNA-template, and the DNA template for transcription needs to include sequences corresponding to (capable of directing synthesis of and encoding) the mRNA species of the mRNA library, i.e., the DNA template has to be a DNA template library comprising DNA species corresponding to the mRNA species in the mRNA library. In addition to the sequences in the mRNA library, the DNA library must include a promotor for RNA transcription, preferably a promotor for SP6 RNA polymerase or T7 RNA polymerase or Syn5 RNA polymerase.

When libraries of bigger sizes are needed, it is desirable to use a split-pool protocol. In such protocol, standard RNA- or DNA-synthesis is carried out until variable regions is to be synthesized. If the codon to be varied originally encodes e.g., leucin, then the oligonucleotide synthesis reaction may split into to four sub-reactions. In each of these sub-reactions, one codon encoding leucin is added (one synonymous codon per sub-reaction to a total of 4 synonymous codons for the 4 sub-reactions). Next, the four sub-reactions are pooled (mixed) and the pooled reaction can then be split before adding the next varied codon position. Obviously, conserved codons are added without splitting the reaction. The principles of split-pool synthesis are well known. An advanced form of split-pool synthesis is used creation of DNA encoded chemical libraries, where a DNA-tag encodes the chemical compound, i.e., the molecules are bifunctional and bidirectional. In a preferred embodiment, DNA-encoded chemical libraries prepared by split-pool synthesis are not part of the present invention.

An alternative to using split-mix synthesis, is to generate trimer phosphoramidites corresponding to all aa codons. When a certain codon position is to be varied during synthesis, e.g., leucine, then the four synonymous trimer phosphoramidite codons encoding leucine is added to the reaction in ratios that will result in the desired ratio of codons. Here, obviously the reaction factors for the various trimer phosphoramidite codons will have to be taken into account. Trimer codons for all aa are commercially available and the person skilled in the art can synthesise synonymous trimer phosphoramidite codons that are not commercially available.

For synthesis of longer DNA templates that may be difficult to synthesize chemically, DNA synthesis may be divided into shorter subsequences that are subsequently assembled to the desired full-length sequence using e.g., PCR tricks. Libraries of short nucleic acids described above are preferred embodiments of such subsequences. As mentioned above, when libraries of short nucleic acids of the invention are used to assemble larger libraries of longer nucleic acids, a plurality of libraries of short nucleic acids are needed, wherein the nucleic acid species of each library of short nucleic acids has a sequence of triplet codons encoding the same polypeptide, but wherein the encoded polypeptide is not the same for different libraries of short nucleic acids. However, the polypeptides of the different libraries of short nucleic acids are preferably part of the same naturally occurring polypeptide.

Subsequences may be assembled using enzymatic or chemical ligation. A preferred method of assembling subsequences is by letting them function as primers and templates on each other in assembly PCR. In this embodiment, the subsequences comprise overlapping sequences so they can anneal to each other to facilitate assembly. And it is preferred that overlapping sequences are in conserved regions of the mRNA library. Not all subsequences used for assembling a longer library of nucleic acid species need to contain variable regions. I.e. they can have a sequence corresponding to the parental mRNA.

### Selecting Variable Regions for the Nucleic Acid Species of the Library

As described above, there is a limit to the length of variable regions in the sequence of triplet codons, since there is an upper limit to diversity based on number of molecules. Thus, for an mRNA with a certain number of codons, it is not possible to vary all codon positions with all synonymous codons.

Even if using just the two best synonymous codons (e.g., corresponding to the most highly expressed tRNAs) for each amino acid, a sequence of 50 triplet codons will lead to a diversity of 2⁵⁰=1.13×10¹⁵. Thus, for longer sequences of triplet codons, it is necessary to select the regions in which to use synonymous codons to create variable regions.

In a preferred embodiment, the variable regions within the sequence of triplet codons are selected based on fragile regions and sites in the mRNA. Thus, if the goal is to improve the stability of a certain mRNA, e.g., the mRNA encoding the spike protein of SARS-COV-2, the mRNA of interest is first synthesised, e.g. with T7 RNA polymerase. Then the mRNA is be incubated under the conditions where it is desired to improve the stability of the mRNA. Degradation of the mRNA is then be followed over time, and the degradation fragments are used to identify fragile regions and sites in the mRNA. Degradation fragments are visualized by gel electrophoresis and isolated from the gel and the sequences of the degradation fragments are obtained using RNA-seq.

When the most fragile regions and sites of the mRNA have been identified, a library of nucleic acid species is created wherein the codons around the fragile regions and/or sites are varied using synonymous codons. In this way, the primary, secondary and tertiary structure around the fragile regions and sites is changed, without changing the amino acid sequence of the polypeptide encoded by the mRNA. Changing the structure around fragile regions and sites can be used prevent the mRNA from breaking at the given region and/or site, hence stabilizing the mRNA. A variable region of e.g., 7 codon positions each with two synonymous codons may introduced at a fragile site, e.g., with the fragile site centrally located in the variable region. This will give a variable region of 256 sequences of which some will have reduced fragility. A variable region of 8 codons each with two synonymous codons will give a variable region of 1024 sequences improving chances of finding sequences with reduced fragility. In one embodiment, the codons of the parental version of the mRNA to be stabilized, are omitted in the variable region to create structure that differ as much as possible from the parental version. Obviously, the variable region around fragile regions and sites can be longer and more than 2 synonymous codons or all synonymous codons for a given amino acid can be used. E.g., 40 codon positions around a fragile site may be varied with two synonymous codons, which give a diversity and library size of 2⁴⁰=1.1×10¹². In a preferred embodiment, varied codon positions are followed by invariable codon positions, e.g., 1 varied position to 1 invariable position, or 1 to 2, 1 to 3 or 1 to 4. In such way, it is possible to introduce diversity over longer sequences.

Fragile regions and sites in an mRNA may be stabilized one at a time, especially if they are separated by a certain distance in the original mRNA sequence, e.g., at least 50 nucleotides or at least 100 nucleotides.

In another embodiment, the original mRNA is divided into shorter fragments of between 100-500 nucleotides that are stabilized individually.

In a preferred embodiment, libraries of stabilized shorter mRNA fragments (or DNA sequences corresponding to the mRNA fragments) are shuffled to create a library of nucleic acids encoding a full-length polypeptide and this library may be used for selecting the most stable full-length mRNAs. After selecting the most stable full-length mRNAs, the process may be started over again by identifying the most fragile sites and regions of the already stabilized mRNAs.

### SECOND ASPECT—METHOD OF SELECTING MRNA SPECIES

A second aspect of the present invention, is a method of selecting mRNA species with a certain characteristic in a mRNA library according to the first aspect of the invention comprising the steps of:
a. Providing a library of mRNA species according the first aspect of the invention
b. Selecting mRNA species which a certain characteristic

### Certain Characteristic—Stability

In a preferred embodiment, the certain characteristic is RNA stability, e.g., chemical stability, or biostability. Thus, the mRNA library is incubated under conditions which will lead to at least some degradation of the mRNA.

This may e.g., be in H₂O, in saline solution, or even solutions that are expected to stabilize RNA, e.g., a pharmaceutical formulation optimized for long shelf life. Divalent cations such as Mg²⁺ may also be included although these often catalyze hydrolysis of RNA.

Also, the temperature is a relevant parameter to adjust, and it could be any temperature at which it is desired to identify mRNA species of improved stability. Thus, the temperature could be 4° C., 20° C., 37° C. or even more. It might also be desired to identify mRNA species with improved stability in frozen condition, e.g., at −20° C. or −80° C. Also, combinations of conditions may be used.

The mRNA library may also be exposed to e.g., serum, plasma or blood to select the mRNA species that have the best stability in those conditions.

The mRNA library may also be transfected into cells to select mRNA species that have the best stability within cells. Alternatively, the mRNA library could be exposed to a cell lysate to select the most stable mRNA species.

Preferably, the mRNA library should be incubated under given conditions for an amount of time that will lead to an enrichment of the more stable mRNA species over less stable mRNA species. It is preferred that the incubation time leads to at least 50% of all mRNAs in the library no longer being fully intact, such as at least 60%, 70%, 80%, 90%, 95%, 99%, 99.9%, 99.99%, 99.999%, 99.9999u % or all mRNAs in the library no longer being fully intact. In a preferred embodiment, at least 0.001% of the mRNAs are still intact, such as at least 0.01% or 0.1%.

### Certain Characteristic—Activity

The certain characteristic could also be capability of being internalized into a cell. In this embodiment, a mRNA library may be incubated with one or more cells, whereafter RNA is purified from the cell(s) to select internalized mRNA species.

The certain characteristic could also be the translational efficiency. In such an embodiment, the mRNA species may be used for mRNA display where the encoded polypeptide is covalently linked to the mRNA species if the mRNA species is translated to the end. Thus, if a given mRNA species is poorly or slowly translated, such mRNA species may be de-selected from a library by only selecting mRNAs that give rise to a full-length polypeptide under given conditions. Selecting such mRNAs may be done using antibodies directed to the encoded polypeptide as the encoded polypeptide will be covalently linked to the mRNA species if the mRNA species is efficiently translated. Another option is to add a tag to the c-terminus of the polypeptide that can be used to select full length proteins such as a GST-tag or a His-tag.

In another preferred embodiment, the certain characteristic is the immune stimulatory activity of the mRNA species of the library, where less immune stimulatory activity is more desired.

### Identification

After selection, selected mRNA species may be identified by sequencing, e.g., by RT-PCR followed by cloning and sequencing or the mRNA species may be sequenced using RNA-seq.

### Iterative Rounds

The selected mRNA species may also be subjected to RT-PCR such as to generate a new DNA library with a promotor for RNA transcription. This DNA library may next be used to generate a second-generation mRNA library enriched in mRNA species that was selected in the previous round. This 2^{nd} generation mRNA library may now be further enriched by performing yet another step of selecting mRNA species which a certain characteristic, where the certain characteristic may be same as in the first round of selection. The certain characteristic could also be different to the selection criteria used in the first round, e.g., stability in a different buffer or at a different temperature. It could be stability in the same buffer, but with a more stringent selection achieved by longer incubation time. It could also be that the selection criteria in the first round was stability under certain conditions and the selection criteria in the second round was translational efficiency.

1.-15. (canceled)

16. A library of nucleic acid species composed of RNA such as mRNA and created by chemical synthesis or in vitro transcription, wherein all nucleic acid species comprises a unique sequence of triplet codons encoding the same polypeptide and wherein the library includes at least 10⁵ nucleic acid species.

17. A library of nucleic acid species according to claim 16, wherein the library includes at least 106 nucleic acid species.

18. The library of any of claim 16, wherein the sequence of triplet codons has a length between 60 and 6000 nucleotides.

19. The library of any of claim 16, wherein the sequence of triplet codons comprises both variable and invariable regions wherein
a. invariable regions are those that are shared between all nucleic acid species of the library and variable regions are those that are not shared between all nucleic acid species of the library and
b. the total length of variable regions within the sequence of triplet codons is no longer than 150 nucleotides and
c. two different synonymous triplet codons are seen as a variable region of 3 nucleotides, even though only one nucleotide differ between the two synonymous codons.

20. The library of claim 16, wherein
a. pseudouridine or 1-methyl-3′-pseudouridylyl is included instead of uridine in the RNA species or
b. 5-methylcytosine is included in the RNA species instead of cytosine.

21. A method of identifying mRNA species with a certain improved characteristic in a mRNA library comprising the steps of:
a. Providing a library of mRNA species according any of claim 16
b. Selecting mRNA species with a certain improved characteristic, wherein the characteristic is selected from the group consisting of: biostability, chemical stability and efficiency of translation.

22. The method of claim 21, wherein the certain improved characteristic is biostability or chemical stability and wherein the library is incubated under conditions which will lead to at least some degradation of the mRNA.

23. The method of claim 22, wherein the library is incubated under conditions for an amount of time that will lead to an enrichment of more stable mRNA species over less stable mRNA species.

24. The method of claim 23, wherein at least 50% of all mRNAs in the library is no longer is fully intact.

25. The method of any of claim 22, wherein the library is incubated in H₂O, saline solution or a pharmaceutical formulation optimized for long shelf life.

26. The method of any of claim 22, wherein the library is exposed to serum, plasma or blood to select mRNA species that have the best stability in those conditions.

27. The method of any of claim 22, wherein the library is transfected into cells to select mRNA species that have the best stability within cells.

28. The method of any of claim 22, wherein mRNA library is be exposed to a cell lysate to select the most stable mRNA species.

29. The method of any of claim 22, further comprising the steps of
a. Reverse transcription and PCR amplification of selected mRNA species to generate a DNA template for RNA transcription
b. RNA transcription using the DNA template generated in the previous step to generate a second-generation mRNA library enriched in mRNA species that was selected in the previous round
c. Performing another step of another step of selecting mRNA species which a certain characteristic using the second-generation mRNA library of the previous step b, wherein the certain characteristic may be same as in the first round of selection or be different to the selection criteria used in the first round.

30. The method of claim 29, wherein selection criteria in the first round was stability under certain conditions and the selection criteria in the second round was translational efficiency.
